(19) United States
(12) Patent Application Publication
(10) Pub. No.: US 2008/0216197 A1
(43) Pub. Date: Sep. 4, 2008

(54) Polypeptide Having Function Related to Pyridoxine Biosynthesis, Polynucleotide Coding the Polypeptide, and Those Use

(75) Inventors: Dong-hee Lee, Busan (KR); In-taek Hwang, Dadjeon (FR); Kwang-yun Cho, Daejeon (KR); Tae-hoon Kim, Busan (KR)

Correspondence Address: INTELLECTUAL PROPERTY LAW GROUP LLP, 12 SOUTH FIRST STREET, SUITE 1205, SAN JOSE, CA, 95113, US

(73) Assignees: Genomine, Inc., Pohang, Kyungbuk (KR); Korean Research Instittute Of Chemcial Technology, Daejon (KR)

(21) Appl. No.: 11/815,314

(22) PCT Filed: Feb. 1, 2006
(86) PCT No.: PCT/KR06/00350

§ 371 Date: Aug. 1, 2007

(30) Foreign Application Priority Data
Feb. 1, 2005 (KR) 10-2005-0008970

(51) Int. Cl. C12N 15/82; C07K 16/00; C07H 21/04; C12Q 1/00; C12N 15/00; C12N 5/00

(52) U.S. Cl. 800/290; 530/350; 536/23.6; 536/24.5; 435/320.1; 435/419; 435/4

(57) ABSTRACT

Disclosed herein are a polypeptide having a pyridoxine biosynthesis-related function, a polypeptide encoding the same, and uses thereof.

### TECHNICAL FIELD

The present invention relates to a polypeptide having a pyridoxine biosynthesis-related function, a polynucleotide encoding the same, and uses thereof.

### BACKGROUND ART

Pyridonxine, the common name of the compound 2-methyl-3-hydroxy-4,5-di(hydroxymethyl)pyridine, belongs to the vitamin B6 family and is essential for the growth of animals and plants (Gregory J F, Ann Rev Nutr 18: 277-296, 1998). In addition to pyridoxine, pyridoxamine and pyridoxal also belong to the vitamin B6 family. These compounds are converted in vivo to pyridoxal-5-phosphate, which is a cofactor in many reactions of amino acid metabolism. Pyridoxal-5′-phosphate is also known to be involved in nitrogen metabolism in all livings.

In plants, there is a pyridoxine biosynthesis pathway, whereas animals, including humans, cannot themselves synthesize pyridoxine due to the lack of the pyridoxine biosynthesis pathway (Dolphin et al., in Vitamin B6 Pyridoxal Phosphate, 1986). Thus, animals must take in pyridoxine from the outside.

The fact that although pyridoxine is essential for the growth of both animals and plants, animals lack a pyridoxine biosynthesis pathway whereas plants can synthesize pyridoxine by themselves has important meaning, implying that if pyridoxine biosynthesis is inhibited, it is possible to effectively suppress the growth of plants without injuring animals.

For this reason, botanists have made a great effort to find polypeptides (enzymes) or polynucleotides (genes) involved in pyridoxine biosynthesis.

Under this background, the present invention has been accomplished.

### DISCLOSURE

### Technical Problem

Therefore, it is an object of the present invention to provide a polypeptide which plays a role in pyridoxine biosynthesis.

It is another object of the present invention to provide a polynucleotide encoding the polypeptide.

It is a further object of the present invention to provide an antisense nucleotide complementary to the polynucleotide.

It is still a further object of the present invention to provide a recombinant vector carrying the polynucleotide and a transformant harboring the recombinant vector.

It is still another object of the present invention to provide a method for suppressing the growth of plants.

It is yet another object of the present invention to provide a method for screening material that suppresses the growth of plants.

It is still yet object of the present invention to provide material that suppresses the growth of plants, obtained using the screening method.

### Technical Solution

In accordance with an aspect of the present invention, a polypeptide which is involved in pyridoxine biosynthesis is provided.

Using primers synthesized on the basis of a putative stress-response protein (GeneBank accession number NM 129380) of Arabidopsis thaliana, a full-length cDNA was obtained. From the base sequence of the cDNA, that is, the base sequence of SEQ. ID. NO. 1, an open reading frame was read to analyze an amino acid sequence, which is listed in SEQ. ID. NO. 2, and calculate the molecular weight of the encoded polypeptide.

A recombinant expression vector carrying the cDNA was inserted into E. coli, and then expressed. The polypeptide thus obtained was found to have the same molecular weight as the calculated weight. Further, the mutant Arabidopsis thaliana, which was transformed with an antisense nucleotide synthesized on the basis of the base sequence of the cDNA, that is, SEQ. ID. NO. 1, was found to be a pyridoxine auxotroph that recovers its phenotype upon pyridoxine treatment, implying that the polypeptide is directly or indirectly involved in pyridoxine biosynthesis.

Therefore, the term “pyridoxine biosynthesis-related function” as used herein means a function essential for pyridoxine biosynthesis and in more detail, an enzymatic function responsible for pyridoxine biosynthesis.

In accordance with the present invention, the polypeptide having a pyridoxine biosynthesis-related function is one of the following polypeptides.

(a) a polypeptide having an amino acid sequence 100% coincident with SEQ. ID. NO 2;

(b) a polypeptide containing a substantial part of the amino acid sequence of SEQ. ID. NO. 2; and

(c) a polypeptide substantially similar to that of (a) or (b).

Herein, the phrase or term “a polypeptide containing a substantial part of the amino acid sequence of SEQ. ID. NO. 2” is defined as a polypeptide containing a part of the amino acid sequence of SEQ. ID. NO. 2 that still has the same pyridoxine biosynthesis-related function as the polypeptide consisting of the amino acid sequence of SEQ. ID. NO. 2. Any polypeptide, as long as it retains the pyridoxine biosynthesis-related function, satisfies the requirement of the present invention, and thus its length or activity is not important. That is, even if lower in activity than the polypeptide of SEQ. ID. NO. 2, any polypeptide that has the pyridoxine biosynthesis-related function may be included within the range of “the polypeptide that contains a substantial part of the amino acid sequence of SEQ. ID. NO. 2”, irrespective of sequence length.

Those who are skilled in the art, that is, those who understand the prior art related to the present invention expect that a deletion or an addition mutant of a polypeptide containing the amino acid sequence of SEQ. ID. NO. 2 will still retains the pyridoxine biosynthesis-related function. As such, a polypeptide which contains the amino acid sequence of SEQ. ID. NO. 2, but from which an N- or C-terminal region has been deleted, is still functional. Generally, it is accepted in the art that even if its N-terminal region or C-terminal region is deleted therefrom, a mutant polypeptide can still retain the function of the intact polypeptide. As a matter of course, if the deleted N- or C-terminal region corresponds to a motif essential for the function of the peptide, the deleted polypeptide loses the function of the intact polypeptide. Nonetheless, the discrimination of such inactive polypeptides from active polypeptides is well known to those skilled in the art. Further, a mutant polypeptide which lacks a portion other than an N- or C-terminal region can still retain the function of the intact polypeptide. Also, those skilled in the art can readily examine whether or not such a deletion mutant still retains the function of the intact polypeptide.

Particularly, in light of the fact that the present invention discloses the base sequence of SEQ. ID. NO. 1 and the amino acid sequence of SEQ. ID. NO. 2 and provides examples in which whether the polypeptide consisting of the amino acid sequence of SEQ. ID. NO. 2 encoded by the base sequence of SEQ. ID. NO. 1 has a pyridoxine biosynthesis-related function was clearly examined, it will be very apparent that those who are skilled in the can examine whether a deletion mutant of the polypeptide comprising the amino acid sequence of SEQ. ID. NO. 2 still functions like the intact polypeptide.

Accordingly, it must be understood in the present invention that “a polypeptide containing a substantial part of the amino acid sequence of SEQ. ID. NO. 2” means any deletion mutant that can be prepared on the basis of the disclosure of the invention by those skilled in the art and that retains the pyridoxine biosynthesis-related function.

The phase “a polypeptide substantially similar to that of (a) or (b)” means a mutant that has at least one substituted amino acid residue but still retains the function of the amino acid sequence of SEQ. ID. NO. 2, that is, the pyridoxine biosynthesis-related function. Likewise, if a mutant in which at least one amino acid residue is substituted still shows the pyridoxine biosynthesis-related function, its activity or substitution percentage is not important. Accordingly, no matter how much lower a mutant polypeptide is in activity than a polypeptide containing the intact amino acid sequence of SEQ. ID. NO. 2, or no matter how much a mutant polypeptide has been substituted with amino acid residues compared to a polypeptide containing the intact amino acid sequence of SEQ. ID. NO. 2, the mutant polypeptide is included within the scope of the present invention as long as it shows the pyridoxine biosynthesis-related function. Even if having at least one amino acid residue substituted for a corresponding residue of the intact polypeptide, a mutant polypeptide still retains the function of the intact polypeptide if the substituted amino acid residue is chemically equivalent to the corresponding one. For instance, when alanine, a hydrophobic amino acid, is substituted with a similarly hydrophobic amino acid, e.g., glycine, or with a more hydrophobic amino acid, e.g, valine, leucine or isoleucine, the polypeptide(s) containing such substituted amino acid residue(s) still retain(s) the function of the intact polypeptide, even if it has lower activity. Likewise, a polypeptide containing substituted amino acid residue(s), resulting from substitution between negatively charged amino acids, e.g., glutamate and aspartate, still retains the function of the intact polypeptide, even if it has lower activity. Also, this is true of a mutant polypeptide in which substitution occurs between positively charged amino acids. For example, a substitution mutant polypeptide, containing lysine instead of arginine, still shows the function of the intact polypeptide even if its activity is lower. In addition, polypeptides which contain substituted amino acid(s) in their N- or C-terminal regions still retain the function of the intact polypeptide. Current technology in the art makes it possible to prepare a mutant polypeptide that retains the pyridoxine biosynthesis-related function of the polypeptide containing the amino acid sequence of SEQ. ID. NO. 2, with at least one amino acid residue substituted therein. Also, those skilled in the art can examine whether a substitution mutant polypeptide still retains the function of the intact polypeptide. Further, because the present invention discloses the base sequence of SEQ. ID. NO. 1 and the amino acid sequence of SEQ. ID. NO. 2 and provides examples in which whether the polypeptide consisting of the amino acid sequence of SEQ. ID. NO. 2 encoded by the base sequence of SEQ. ID. NO. 1 has a pyridoxine biosynthesis-related function was clearly examined, it will be very apparent that “the polypeptide substantially similar to that of (a) or (b)” can be readily prepared by those who are skilled in the art. Accordingly, the “polypeptide substantially similar to that of (a) or (b)” is understood to include all polypeptides that have the pyridoxine biosynthesis-related function in spite of the presence of at least one substituted amino acid therein. Nevertheless, a polypeptide which shares higher homology with the amino acid sequence of SEQ. ID. NO. 2 is more preferable from the point of view of activity. Useful is a polypeptide that shows 60% or higher homology with the wild-type polypeptide, with the best preference for 100% homology.

In more detail, more preferable are sequence homologies of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99%, in ascending order of preference.

Because “the polypeptide substantially similar to that of (a) or (b) includes polypeptides substantially similar to “the polypeptide containing a substantial part of the amino acid sequence of SEQ. ID. NO. 2” as well as polypeptides substantially similar to “the polypeptide having an amino acid sequence 100% coincident with SEQ. ID. NO. 2”, the above description is true both for polypeptides substantially similar to “the polypeptide having an amino acid sequence 100% coincident with SEQ. ID. NO. 2” and for polypeptides substantially similar to “the polypeptide containing a substantial part of the amino acid sequence of SEQ. ID. NO. 2”.

In accordance with another aspect of the present invention, an isolated polynucleotide encoding the above-mentioned polypeptide is provided. Herein, “the above-mentioned polypeptide” is intended to include not only the polypeptide having the amino acid sequence of SEQ. ID. NO. 2, polypeptides containing a substantial part of the amino acid sequence of SEQ. ID. NO. 2, and polypeptides substantially similar to these peptides, but also all polypeptides that retain the pyridoxine biosynthesis-related function in the preferred embodiments. Therefore, the polynucleotide of the present invention includes an isolated polynucleotide encoding a polypeptide that has the pyridoxine biosynthesis-related function and contains the entire amino acid sequence of SEQ. ID. NO. 2 or a substantial part of the amino acid sequence thereof, and an isolated polynucleotide encoding a polypeptide substantially similar to such polypeptides. Furthermore, the polynucleotide of the present invention includes all isolated polynucleotides encoding polypeptides which share homology with the amino acid sequence of SEQ. ID. NO. 2.

If an amino acid sequence is revealed, a polynucleotide encoding the amino acid sequence can be readily prepared on the basis of the amino acid sequence by those skilled in the art.

In the present invention, the phrase “the isolated polynucleotide” as used herein is intended to include all chemically synthetic polynucleotides, isolated polynucleotides from living bodies, especially Arabidopsis thaliana), and polynucleotides containing modified nucleotides, whether single or double strand RNA or DNA. Accordingly, cDNAs, chemically synthetic polynucleotides, and gDNAs isolated from living bodies, especially Arabidopsis thaliana fall into the range of “the isolated polynucleotide”. On the basis of the amino acid sequence of SEQ. ID. NO. 2, and the base sequence of SEQ. ID. NO. 1 encoding the amino acid sequence, and technology known in the art, the preparation of corresponding cDNAs and chemically synthetic polynucleotides and the isolation of gDNA can be readily achieved by those who are skilled in the art.

In accordance with a further aspect of the present invention, a polynucleotide that contains or is substantially similar to part of the base sequence of SEQ. ID. NO. 1 is provided. Herein, the phrase “a polynucleotide that contains part of the base sequence of SEQ. ID. NO. 1” means a polynucleotide that has a sequence long enough to identify and/or isolate a gene having the pyridoxine biosynthesis-related function in living bodies, especially Arabidopsis thaliana. The phrase “a polynucleotide that is substantially similar to part of the base sequence of SEQ. ID. NO. 1” means a polynucleotide that contains at least one substituted nucleotide residue, compared to the base sequence of SEQ. ID. NO. 1, and has sequence-dependent binding ability sufficient to identify and/or isolate a gene having pyridoxine biosynthesis-related function in living bodies including Arabidopsis thaliana.

As long as the base sequence of SEQ. ID. NO. 1 is disclosed, the identification and/or isolation of a gene having the pyridoxine biosynthesis-related function in Arabidopsis thaliana or other organisms can be readily carried out by those skilled in the art.

Accordingly, the polynucleotide of the present invention is intended to include all polynucleotides which have a sequence length or sequence-dependent binding power sufficient to identify and/or isolate a gene having the pyridoxine biosynthesis-related function in living bodies including Arabidopsis thaliana, irrespective of the length and sequence homology to the base sequence of SEQ. ID. NO. 1.

In order to be used as a probe for examining whether or not an unknown gene has the same base sequence as that of a known gene or for isolating an unknown gene, a polynucleotide is generally known to have to have 30 or more consequent nucleotide residues. Thus, the polynucleotide of the present invention preferably includes 30 or more consequent nucleotide residues out of the base sequence of SEQ. ID. NO. 1. However, a poly(or oligo)peptide consisting of 30 or fewer consequent nucleotide residues out of the base sequence of SEQ. ID. NO. is still included within the scope of the present invention. The reason is that the poly(or oligo)nucleotide, although short, is sufficient to identify and/or isolate a gene having the pyridoxine biosynthesis-related function from Arabidopsis thaliana or other organisms if it shares 100 homology with part of the base sequence of SEQ. ID. NO. 1 and the identification and/or isolation condition (buffer pH, concentration, etc.) is stringent. Based on the disclosure of the present invention, herein, those skilled in the art can readily construct and detect a polynucleotide which is 30 or fewer bases long in order to identify and/isolate a gene having the pyridoxine biosynthesis-related function from Arabidopsis thaliana or other organisms and can readily identify and/or isolate a gene having the pyridoxine biosynthesis-related function from Arabidopsis thaliana or other organisms using the constructed polynucleotide.

In accordance with still a further aspect of the present invention, an antisense nucleotide able to complementarily bind to the above-mentioned polynucleotide is provided.

The antisense nucleotide is intended to include all poly(or oligo)nucleotides that complementarily bind to the above-mentioned polynucleotide to inhibit transcription (when the polynucleotide is DNA) or the translation (when the polynucleotide is RNA).

If the antisense nucleotide can complementarily bind to the polynucleotide encoding the polypeptide having the pyridoxine biosynthesis-related function to inhibit the transcription or translation of the polynucleotide (respectively DNA or RNA), its length or homology to a complementary sequence is not important. A polynucleotide, even if short, e.g., 30 bases long, can function as an antisense nucleotide as long as it shares 100% homology with a sequence complementary to the gene of interest (DNA or RNA) and stringent conditions including buffer concentration and pH are observed. Additionally, although it does not share 100% homology with a complementary sequence of the gene of interest, a polynucleotide may be used as an antisense nucleotide if it has a suitable length.

Therefore, it should be noted that as long as it can inhibit the transcription or translation of a gene of interest, any poly(or oligo)nucleotide is included in the range of the antisense nucleotide of the present invention, irrespective of length and homology to a complementary sequence. On the basis of the base sequence of SEQ. ID. NO. 1 and the amino acid sequence of SEQ. ID. NO. 2, those skilled in the art can readily determine the length and homology necessary for an antisense nucleotide and prepare such an antisense nucleotide using current technology.

Preferable is the antisense nucleotide the complete or partial sequence of which is complementary to a length of the base sequence of SEQ. ID. NO. 1. In light of the previous description, herein, the phrase “complementary to a length of the base sequence of SEQ. ID. NO. 1” should be understood to be long enough to bind to DNA comprising the base sequence of SEQ. ID. NO. 1 or to an RNA transcripted from the DNA so as to inhibit the transcription or translation of the polynucleotide.

In accordance with still another aspect of the present invention, a recombinant vector containing the above-mentioned polynucleotide therein and a transformant carrying the recombinant vector are provided.

In the following examples, a polynucleotide, based on the base sequence of SEQ. ID. NO. 1, coding for a polypeptide having a pyridoxine biosynthesis-related function was inserted into pCAL-n (Stratagene, USA) to construct the recombinant vector pCAtPDX5. E. coli was transformed with the recombinant vector and then allowed to express the polypeptide from the polynucleotide. The molecular weight of the expressed polypeptide was measured to be identical to that inferred from the ORF of the base sequence of SEQ. ID. NO. 1.

Preferably in consideration of the embodiments, the recombinant vector is pCAtPDX5 and the transformant is E. coli transformed with the recombinant vector.

In accordance with yet another aspect of the present invention, a method for suppressing the growth of plants is provided. The method comprises suppressing the expression or activity of the polypeptide, based on the amino acid sequence of SEQ. ID. NO. 2 or a similar amino acid sequence, having the pyridoxine biosynthesis-related function.

As described above, pyridoxine is a vitamin essential for the growth of both plants and animals, and its biosynthesis pathway exists in plants, but not in animals. Thus, the suppression of the expression or activity of the polypeptide having the pyridoxine biosynthesis-related function leads to the suppression of the growth of plants without injuring animals. When an antisense nucleotide complementary to the base sequence of SEQ. ID. NO. 1 is introduced into Arabidopsis thaliana, as will be understood later, the growth of the transformed Arabidopsis thaliana is found to be delayed. Thus, the method for suppressing the growth of plants in accordance with the present invention can be accomplished by suppressing the expression or activity of the polypeptide having the pyridoxine biosynthesis-related function.

Herein, the phrase “a polypeptide consisting of an amino acid sequence similar to that of SEQ. ID. NO. 2” is intended to include all polypeptides that are homologs of the polypeptide of SEQ. ID. NO. 2, with the retention of the pyridoxine biosynthesis-related function, and are different in amino acid sequence from the polypeptide of SEQ. ID. NO. 2 due to evolutionary differences among plants. In the method for suppressing the growth of plants in accordance with the present invention, the plants include all types of plants as well as Arabidopsis thaliana although the polypeptide consisting of the amino acid sequence of SEQ. ID. NO. 2 was isolated from Arabidopsis thaliana. More preferable from the point of view of activity is a polypeptide consisting of an amino acid sequence similar to that of SEQ. ID. NO. 2 which shares higher homology with the amino acid sequence of SEQ. ID. NO. 2. Useful is a polypeptide that shows 60% or higher homology with the wild-type polypeptide, with the best preference for 100% homology.

In more detail, more preferable are sequence homologies of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99%, in ascending order of preference.

The suppression of polypeptide expression can be achieved using various methods well known in the art, including antisense nucleotide introduction, gene deletion, gene insertion, T-DNA introduction, homologous recombination, transposon tagging, and RNA silencing with siRNA (small interfering RNA).

In the following examples, antisense nucleotide introduction was utilized to suppress the growth of plants. In detail, an antisense nucleotide to a polynucleotide consisting of the base sequence of SEQ. ID. NO. 1 was prepared and inserted into a vector. The recombinant vector (pSEN-antiAtPDX5) thus constructed was introduced into Agrobacterium tumefaciens which was then transfected into Arabidopsis thaliana. Seeds from the resulting mutant Arabidopsis thaliana were found to grow in a significantly delayed manner (see Example 3).

In the method for suppressing the growth of plants, an antisense nucleotide complimentary to part of the base sequence of SEQ. ID. NO. 1 is preferably introduced into plants. More preferably, a transformant harboring a recombinant vector carrying the antisense nucleotide is introduced into plants. Most preferably, the transformant is the Agrobacterium tumefaciens transformed with the recombinant vector. Herein, the phrase “complementary to part of the base sequence of SEQ. ID. NO. 1” has the same meaning as in the description of the antisense nucleotide.

Generally, an antisense nucleotide is known to bind to a target nucleotide in nucleic acids (RNA or DNA) to suppress the function or synthesis of the nucleic acids. With the ability to hybridize both RNA and DNA, an antisense nucleotide corresponding to a target gene can inhibit the expression of the target gene in the transcription or translation level thereof.

Accordingly, the suppression of the expression or activity of a polypeptide consisting of the amino acid sequence of SEQ. ID. NO. 2 or a similar amino acid sequence results in the suppression of the growth of plants.

Taking advantage of the presence of the pyridoxine biosynthesis pathway only in plants, the method for suppressing the growth of plants according to the present invention does not injure humans or animals.

In accordance with yet still another aspect of the present invention, a method for screening a material suppressive of the growth of plants is provided. This method comprises detecting a material that suppresses the expression or activity of the polypeptide consisting of the amino acid sequence of SEQ. ID. NO. 2 or a similar amino acid sequence and having the pyridoxine biosynthesis-related function.

Herein, the phrase “the polypeptide consisting of the amino acid sequence of SEQ. ID. NO. 2 or a similar amino acid sequence” has the same meaning as in the description of the method for suppressing the growth of plants.

For the same reason as in the description of the method for suppressing the growth of plants, the material suppressive of the expression of the polypeptide is preferably an antisense nucleotide complementary to part of the base sequence of SEQ. ID. NO. 1, more preferably a transformant harboring a recombinant vector carrying the antisense nucleotide, and still more preferably Agrobacterium tumefaciens transformed with the recombinant vector. Herein, the phrase “complementary to a part of the base sequence of SEQ. ID. NO. 1” has the same meaning as in the description of the antisense nucleotide.

In accordance with yet still an additional aspect of the present invention, a material suppressive of the growth of plants, obtained through the screening method, is provided.

As such, an antisense nucleotide complementary to part of the base sequence of SEQ. ID. NO. 1, a recombinant vector carrying the antisense nucleotide, and Agrobacterium tumefaciens transformed with the recombinant vector may be exemplified.

### ADVANTAGEOUS EFFECTS

As described above, the present invention provides a polypeptide having a pyridoxine biosynthesis-related function, a polynucleotide encoding the polypeptide, an antisense nucleotide complementary to the polynucleotide, a recombinant vector carrying the polynucleotide, a transformant harboring the recombinant vector, a method for suppressing the growth of plants, a method for screening material that suppresses the growth of plants, and material that suppresses the growth of plants.

### DESCRIPTION OF DRAWINGS

FIG. 1 is a photograph showing the results of SDS-PAGE analysis for proteins from E. coli transformed with a recombinant vector carrying a polynucleotide encoding a polypeptide having a pyridoxine biosynthesis-related function, particularly, a polynucleotide consisting of the base sequence of SEQ. ID. NO. 1 and proteins from a control.

FIG. 2 is a schematic diagram showing the structure of pSEN into which a polynucleotide encoding a polypeptide having a pyridoxine biosynthesis-related function, particularly a polypeptide consisting of the base sequence of SEQ. ID. NO. 1, is to be inserted in an antisense direction.

FIG. 3 is a schematic diagram showing the structure of the recombinant vector pSEN-antiAtPDX5 prepared by inserting a polynucleotide encoding a polypeptide having a pyridoxine biosynthesis-related function, particularly a polypeptide consisting of the base sequence of SEQ. ID. NO. 1, into the vector pSEN in an antisense direction.

FIG. 4 is a photograph showing mutant Arabidopsis thaliana grown from T1 seeds of Arabidopsis thaliana transformed with the vector pSEN of FIG. 2 and the recombinant vector pSEN-antiAtPDX5 of FIG. 3.

FIG. 5 is a photograph showing the mutant Arabidopsis thaliana grown from the T2 seeds of the Arabidopsis thaliana transformed with the recombinant vector pSEN-antiAtPDX5 of FIG. 3.

FIG. 6 is a photograph showing the result of electrophoresis of the RT-PCR products using the transcripts of the polynucleotide obtained from mutant Arabidopsis thaliana grown from T2 seeds of Arabidopsis thaliana transformed with the recombinant vector pSEN-antiAtPDX5 of FIG. 3 and the polynucleotide consisting of the base sequence of SEQ. ID. NO. 1 of a wild-type recombinant vector.

FIG. 7 is a photograph showing Arabidopsis thaliana which has been grown in a pyridoxine-supplemented medium from the T2 seeds of the mutant Arabidopsis thaliana transformed with the recombinant vector pSEN-antiAtPDX5 of FIG. 3.

### BEST MODE

A better understanding of the present invention may be obtained through the following examples which are set forth to illustrate, but are not to be construed as the limit of the present invention.

### Example 1

### Isolation of a Gene Encoding a Polypeptide Having a Pyridoxine Biosynthesis-Related Function from Arabidopsis thaliana

A screening process was performed for isolating a gene, encoding a polypeptide having a pyridoxine biosynthesis-related function, from Arabidopsis thaliana.

### Example 1-1

Cultivation and Nurturance of Arabidopsis thaliana

Arabidopsis thaliana was cultured in soil in pots or in an MS medium (Murashige and Skoog salts, Sigma, USA) containing 2% sucrose (pH 5.7) and 0.8% agar in Petri dishes. All of the MS media used in the present invention were free of B6 family (pyridoxine, etc.). When using pots, the plants were cultivated at 22° C. under a light-dark cycle of 16/8 hours in a growth chamber.

### Example 1-2

### RNA Isolation and cDNA Library Construction

In order to construct Arabidopsis thaliana cDNA libraries, first, RNA was isolated from Arabidopsis thaliana leaves in various stages of differentiation using a TRI reagent (Sigma, USA). poly(A)+ RNA was purified from the isolated total RNA using an mRNA purification kit (Pharmacia, USA) according to the enclosed instructions for the protocol. Double-stranded cDNA was prepared from the poly(A)+ RNA with the aid of a cDNA synthesis kit (Time Saver cDNA synthesis kit, Pharmacia, USA), with NotI-(dT)₁₈ serving as a primer.

### Example 1-3

### Isolation of a Gene Encoding a Polypeptide Having a Pyridoxine Biosynthesis-Related Function

Based on the amino acid sequence of a putative stress-response protein (GeneBank accession number NM 129380) of Arabidopsis thaliana, a sense primer, represented by SEQ. ID. NO. 3, containing an XbaI site, and an antisense primer, represented by SEQ. ID. NO. 4, containing a BglII site were synthesized. Using these two primers, a full length cDNA was amplified through PCR (polymerase chain reaction) from the cDNA libraries constructed in Example 2.

The cDNA was analyzed to have a 930 bp ORF comprised of one exon encoding a polypeptide consisting of 309 amino acid residues with a molecular weight of about 32.8 kDa and was called AtPDX5 (Arabidopsis thaliana pyridoxine biosynthesis protein 5). The protein AtPDX5 encoded by the gene was found to have an isoelectric point of 5.8 (hereinafter, genes are represented in italics, e.g., “AtPDX5” or “AtPDX5 gene”, proteins as “AtPDX5” or “AtPDX5 protein”).

In the amino acid sequence inferred from AtPDX5, an SOR/SNZ family domain and an SNZ1 domain were found to be located at amino acid positions from 20 to 227 and at amino acid positions from 17 to 307, respectively. Because proteins with such domains are known to have an enzymatic function involved in the pyridoxine biosynthesis pathway, an Arabidopsis thaliana mutant was created to examine whether the polynucleotide of the present invention is directly implicated in the pyridoxine biosynthesis pathway.

### Example 2

### Purification of AtPDX5 Protein from E. coli

In Arabidopsis thaliana, the expression of the AtPDX5 protein was induced. In this regard, full-length cDNA was amplified and isolated from the cDNA libraries of Example 1-2 through PCR using a sense primer, represented by SEQ. ID. NO. 5, containing a BglII site, and an antisense primer, represented by SEQ. ID. NO. 6, containing an XhoI site. The PCR product thus obtained was cloned between the BamHI site (BglII compatible end ligation site) and the XhoI site of a pCAL-n vector (Stratagene, USA) to construct a recombinant vector, called pCAtPDX5. The pCAL-n vector is advantageous in that the protein expressed therefrom can be readily separated by calmodulin resin because it has a calmodulin-binding peptide tag.

The pCAtPDX5 recombinant vector was introduced into E. coli BL21-Gold(DE3) (Stratagene, USA) which was then cultured at 37° C. in an LB (Luria-Bertani) broth (USB, USA) in the presence of 100 μg/ml ampicillin to an O.D.600 of 0.7 with stirring at 150 rpm.

In order to induce the intracellular expression of the target protein, IPTG (isopropyl-D-thiogalactoside) was added in a final concentration of 1 mM to the suspension, followed by incubation for an additional 2 hours. The cells were washed with 50 mM-potassium phosphate buffer (pH 7.0) containing 50 mM MgSO₄ and 0.4M NaCl and the cell pellet, obtained by centrifugation at 4,000×g for 15 minutes, was stored at −20° C.

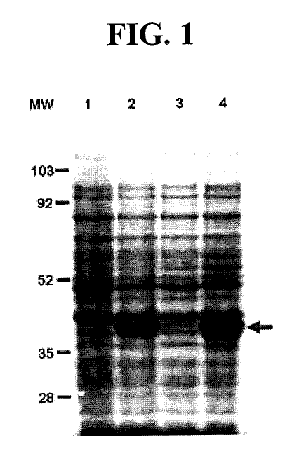

The expression of the protein was examined by SDS-PAGE using a lysate from E. coli transformed with the pCAtPDX5 recombinant vector. The result is given in FIG. 1. A lysate from the E. coli transformed with the pCAtPDX5 recombinant vector was found to contain a fused protein about 37 kDa in size (molecular weight of the protein expressed from the AtPDX5 gene 32.8 kDa+molecular weight of the calmodulin-binding protein 4 kDa) as measured by SDS-PAGE. In contrast, no protein having such a size was found in the lysate of control E. coli (E. coli transformed with pCAL-n vector). In FIG. 1, a 37 kDa fusion protein (molecular weight of the protein expressed from the AtPDX5 gene 32.8 kDa+molecular weight of the calmodulin-binding protein 4 kDa) is indicated by the arrow (←). Lysates from the control E. coli were run on lanes 1 and 3 while lysates from colony-1 and colony-2 of the E. coli transformed with a recombinant vector carrying the AtPDX5 gene were electrophoresed on lanes 2 and 4, respectively.

### Example 3

### Preparation and Characterization of Arabidopsis thaliana Mutant Harboring Antisense Construct Complementary to AtPDX5 Gene

### Example 3-1

### Preparation of Arabidopsis thaliana Mutant Harboring Antisense Construct Complementary to AtPDX5 Gene

To examine physiological properties of the protein isolated in Example 2, the AtPDX5 gene was introduced in the antisense direction into Arabidopsis thaliana to suppress the expression of the AtPDX5 transcript.

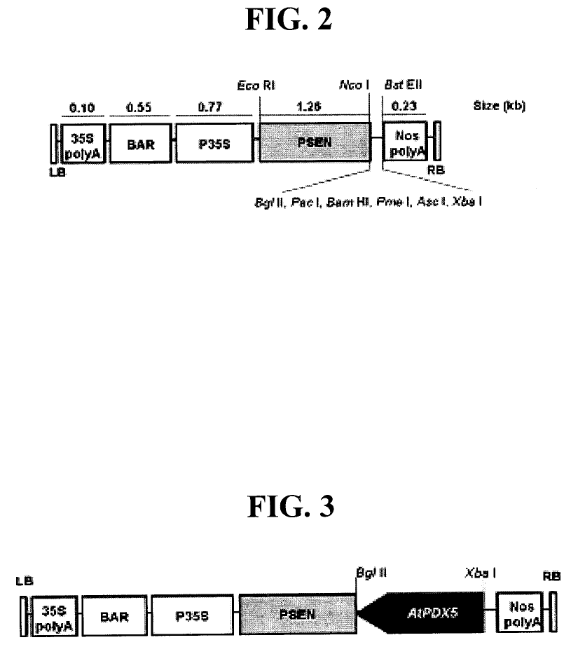

AtPDX5 cDNA was amplified from the cDNA libraries of Arabidopsis thaliana through PCR using a sense primer, represented by SEQ. ID. NO. 3, containing an XbaI site, and an antisense primer, represented by SEQ. ID. NO. 4, containing a BglII site. The PCR product was digested with restriction enzymes BglII and XbaI and inserted in an antisense direction into the pSEN vector, under the control of a senl promoter, a stress or senescence-associated gene, to construct a recombinant vector, named pSEN-antiAtPDX5 harboring an antisense construct complementary to the AtPDX5 gene. Since the senl promoter shows specificity for the genes expressed according to growth stage, the recombinant vector pSEN-antiAtPDX5 can prevent plants from dying in a germination stage. FIGS. 2 and 3 respectively show the structures of the pSEN vector and the pSEN-antiAtPDX5 recombinant vector prepared by introducing the AtPDX5 gene in an antisense direction into the pSEN vector. In FIGS. 2 and 3, BAR stands for a bar gene (phosphinothricin acetyltransferase gene) conferring Basta resistance, RB for a right border, LB for a left border, P35S for a CaMV 35S RNA promoter, 35S poly A for CaMV 35S RNA poly A, PSEN for a senl promoter, and Nos polyA for nopaline synthase gene polyA.

The pSEN-antiAtPDX5 recombinant vector was introduced into Agrobacterium tumefaciens using an electroporation method. The transformed Agrobacterium strain was cultured at 28° C. to an O.D.₆₀₀ of 1.0, followed by harvesting cells by centrifugation at 25° C. at 5,000 rpm for 10 min. The cell pellet thus obtained was suspended in an infiltration medium (1× MS SALTS, 1× B5 vitamin, 5% sucrose, 0.005% Silwet L-77, Lehle Seed, USA) until O.D.₆₀₀ reached 2.0. Four week-old Arabidopsis thaliana was immersed in the Agrobacterium suspension in a vacuum chamber and allowed to stand for 10 min under a pressure of 10⁴ Pa. Thereafter, the Arabidopsis thaliana was placed for 24 hours in a polyethylene bag. The Arabidopsis thaliana was grown to obtain seeds (T1). Wild-type Arabidopsis thaliana or Arabidopsis thaliana harboring the pSEN vector (the antisense AtPDX5 gene was absent) was used as a control.

### Example 3-2

### Characterization of Transformed T1 and T2 Arabidopsis thaliana

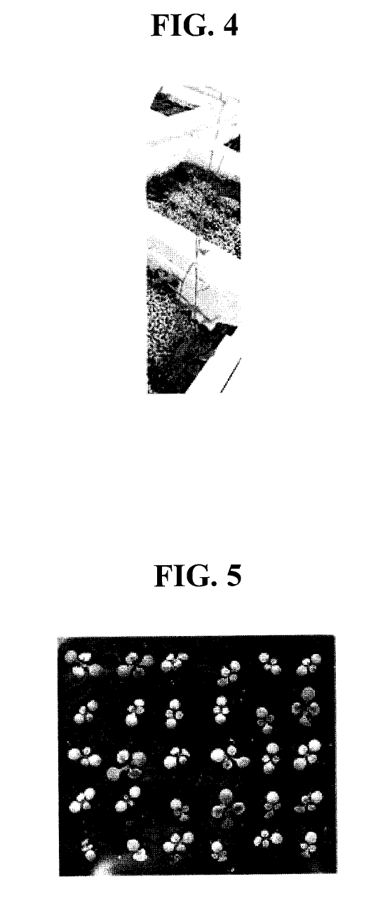

After being immersed in a 0.1% Basta herbicide solution (Kyung Nong Co. Ltd., Korea) for 30 min, seeds from the Arabidopsis thaliana transformed in Example 3-1 were cultured. A Basta herbicide was applied five times to each pot in which the transformed Arabidopsis thaliana grew, and observation was made of the growth pattern of the Arabidopsis thaliana in each pot. Compared to the control (Arabidopsis thaliana harboring a pSEN vector), the Arabidopsis thaliana transformed with the pSEN-antiAtPDX5 recombinant vector was found to grow in a significantly retarded pattern, with etiolation of the leaves, siliques, and stems (FIG. 4). In addition, the potent antisense effect on the gene of the present invention caused death of the plant transformant as well as growth suppression and etiolation.

The phenotype of Arabidopsis thaliana transformed with an antisense construct of the AtPDX5 gene was examined. T2 seeds were obtained from the T1 line of the transformed Arabidopsis thaliana. For this, 30 T2 seeds, which had been subjected to low temperature treatment (4° C.) for 3 days, were cultured in a Petri dish containing an MS medium (30 seeds/Petri dich). After 10 days' cultivation, only five plants had the phenotype of wild-type Arabidopsis thaliana while the remainder 25 individuals were observed to grow in a retarded pattern, with etiolation occurring throughout all leaves (FIG. 5).

To examine whether or not the phenotype had a 3:1 (mutant:wild type) segregation ratio with regard to one copy of the transgene, the plants grown in the Petri dishes were treated with 12.5 mg/L PPT (phosphinothricin, Duchefa, Netherlands). While the five plants having a wild-type phenotype were converted to a fatal phenotype, the other 25 plants remained unchanged, that is, showed etiolation and retarded growth.

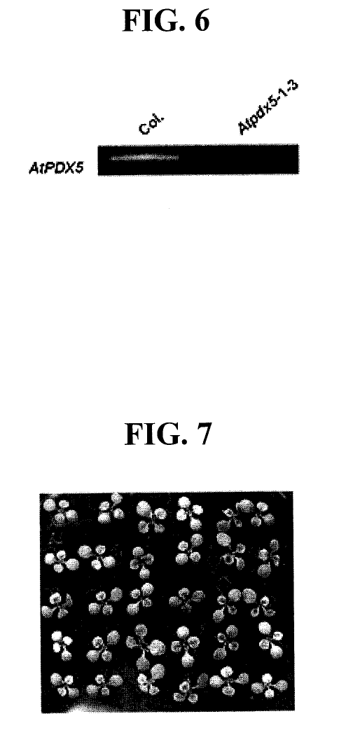

There was a need to examine whether the phenotypic properties of the transformed Arabidopsis thaliana came from a change in the expression of the AtPDX5 gene. RT-PCR was performed using the sense primer of SEQ. ID. NO. 3 and the antisense primer of SEQ. ID. NO. 4, with the RNA purified as in Example 1-2 serving as a template. The PCR product thus obtained was run on agarose gel in the presence of an electric field so as to compare levels of transcripts between the wild-type Arabidopsis thaliana and the mutant Arabidopsis thaliana, selected with PPT treatment, having the phenotype of growth delay and etiolation. A significant decrease of AtPDX5 gene expression was observed in the mutant Arabidopsis thaliana (Atpdx5-1-3) compared to the wild-type (Col.) (FIG. 6), supporting the fact that the suppression of AtPDX5 gene expression leads to the phenotype of growth retardation and etiolation and thus implying that the gene according to the present invention plays an important role in plant development.

The analysis of the AtPDX5 domain led to the inference that the AtPDX5 gene might have an enzymatic function involved in the pyridoxine biosynthesis pathway. To examine this, T2 plants of the mutant Arabidopsis thaliana were cultured in Petri dishes containing a 2.5 mg/L pyridoxine-HCl (Sigma, USA)-supplemented MS medium (30 seeds/Petri dish). Although slight etiolation was observed, there was no significant difference in phenotype, such as growth delay, between the mutant Arabidopsis thaliana and the wild-type (FIG. 7). In addition, the etiolation of the mutant plants was believed to be attributed to a low content of pyridoxine in the medium. Based on the fact that the phenotype recovery was induced by the addition of pyridoxine, the AtPDX5 gene is concluded to be directly responsible for pyridoxine biosynthesis. As described hereinbefore, T2 plants of the mutant Arabidopsis thaliana have the phenotype properties of significant growth delay, etiolation throughout leaves, and death in an early stage, and the mutant Arabidopsis thaliana can have the same phenotype as that of the wild-type in the presence of pyridoxine.

Taken together, the data obtained thus far in accordance with the present invention indicate that the plants transformed with an antisense construct of the AtPDX5 gene are pyridoxine auxotrophs and that the gene of the present invention will be useful in the development of novel plant growth regulators or herbicides.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### SEQUENCE LIST PRETEXT

Sequence list Attached

1. A polypeptide having a pyridoxine biosynthesis-related function, selected from the group consisting of:
(a) a polypeptide having an amino acid sequence 100% coincident with SEQ. ID. NO. 2;
(b) a polypeptide containing a substantial part of the amino acid sequence of SEQ. ID. NO. 2; and
(c) a polypeptide substantially similar to that of (a) or (b).

2. A polynucleotide, encoding the polypeptide of claim 1.

3. An antisense nucleotide, complementary to the polynucleotide of claim 2.

4. A recombinant vector carrying the polynucleotide of claim 2.

5. A transformant harboring the recombinant vector of claim 4.

6. A method for suppressing the growth of plants, comprising the step of suppressing the expression or activity of a polypeptide having a pyridoxine biosynthesis-related function, the polypeptide having an amino acid sequence 100% coincident with or similar to SEQ. ID. NO. 2.

7. The method as defined in claim 6, wherein the suppressing step comprises the introduction of the antisense nucleotide of claim 3 into the plants.

8. The method as defined in claim 6, wherein the suppressing step is carried out using a technique selected from the group consisting of gene deletion, gene insertion, T-DNA introduction, homologous recombination, transposon tagging, RNA silencing with siRNA, and combinations thereof.

9. A method for screening material suppressive of the growth of plants, comprising the step of detecting material suppressive of the expression or activity of a polypeptide, based on an amino acid sequence 100% coincident with or similar to SEQ. ID. NO. 2, having a pyrixodine biosynthesis-related function.

10. A material suppressive of the growth of plants, obtained using the method of claim 9.

11. The material as defined in claim 10, wherein the material is selected from a group consisting of the antisense nucleotide of claim 3, a recombinant harboring the antisense nucleotide vector of claim 3, and Agrobacterium tumefaciens transformed with a recombinant vector harboring the antisense nucleotide of claim 3.


SEQUENCE LISTING (6 sequences)

SEQ ID NO: 1 - DNA, 1039 bp, Arabidopsis thaliana
tctcaaaaccctagaaaaaatggcaggaaccggagttgtggcggtgtacggcgaaggagccatgacggagacgaaacagaaatctcccttctccgtgaaagttggtctcgctcagatgcttcgtggcggtgtaatcatggatgtcgtcaacgcagagcaagctcgaatcgctgaagaagctggcgcatgcgccgtgatggctcttgaacgtgttcccgccgatattcgagctcaaggcggtgttgctcgaatgagcgatccagagatgatcaaagaaatcaaaaacgccgtgacgattccggtgatggcgaaagctagaattggtcatttcgttgaagctcagatcctggaagcaatcggagttgattacgtcgacgagagtgaagttctcactctcgccgacgaagataatcacatcaacaaacataatttcaaaatcccttttgtttgtggatgtaggaatctcggtgaagctttaaggcggatccgtgaaggagccgccatgataagaaccaaaggtgaggctggaactggtaacgttgttgaagccgttaggcacgtgaggagtgtgaacggagctattcggttacttagaagcatggacgatgacgaggttttcacttacgcgaaaaagatcgctgcgccgtatgatttggttgtgcagactaaggagcttgggaggttaccggtggttcagttcgctgctggaggagtggcgacgccggcggatgcggcgttgatgatgcagttgggatgtgatggagtgtttgttgggtcgggtgttttcaagagtggagatccggtgaagagggctaaggctattgttcaggcggttacgaattatagagacgcggcggtgttggcggaggtgagctgtggtttaggtgaagccatggttggtcttaatttggatgataaggttgagaggttcgctagtcgttctgagtaaccaatcaaatttcagatgtttttatccataacgtttgtcactttaatatgtatccacaaaccaatattctcgatttattcaagattttat

SEQ ID NO: 2 - protein, 309 aa, Arabidopsis thaliana
MAGTGVVAVYGEGAMTETKQKSPFSVKVGLAQMLRGGVIMDVVNAEQARIAEEAGACAVMALERVPADIRAQGGVARMSDPEMIKEIKNAVTIPVMAKARIGHFVEAQILEAIGVDYVDESEVLTLADEDNHINKHNFKIPFVCGCRNLGEALRRIREGAAMIRTLGEAGTGNVVEAVRHVRSVNGAIRLLRSMDDDEVFTYAKKIAAPYDLVVQTKELGRLPVVQFAAGGVATPADAALMMQLGCDGVFVGSGVFKSGDPVKRAKAIVQAVTNYRDAAVLAEVSCGLGEAMVGLNLDDKVERFASRSE

SEQ ID NO: 3 - DNA, 28 bp, Artificial
  Sense primer
tctagaatggcaggaaccggagttgtgg

SEQ ID NO: 4 - DNA, 31 bp, Artificial
  Antisense primer
agatctttatggataaaaacatctgaaattt

SEQ ID NO: 5 - DNA, 28 bp, Artificial
  Sense primer
agatctatggcaggaaccggagttgtgg

SEQ ID NO: 6 - DNA, 31 bp, Artificial
  Antisense primer
ctcgagttatggataaaaacatctgaaattt